(19) United States
(12) Patent Application Publication
(10) Pub. No.: US 2017/0369394 A1
(43) Pub. Date: Dec. 28, 2017

(54) ISOPARAFFIN-OLEFIN ALKYLATION

(71) Applicant: ExxonMobil Research and Engineering Company, Annandale, NJ (US)

(72) Inventors: Jihad M. DAKKA, Whitehouse Station, NJ (US); Matthew S. METTLER, Somerville, NJ (US); Charles M. SMITH, Princeton, NJ (US); Ivy D. JOHNSON, Lawrenceville, NJ (US); Jeffrey M. FITT, The Woodlands, TX (US)

(21) Appl. No.: 15/610,729

(22) Filed: Jun. 1, 2017

Related U.S. Application Data
(60) Provisional application No. 62/353,675, filed on Jun. 23, 2016.

(51) Int. Cl. C07C 2/58; B01J 37/06; C07C 7/00; C10L 10/10; C10L 1/16; B01J 29/70; B01J 37/00; B01J 37/08

(52) U.S. Cl. CPC C07C 2/58; B01J 37/0018; B01J 37/06; B01J 37/08; C10L 10/10; C10L 1/1608; B01J 29/7038; C07C 7/00; C07C 2521/08; C07C 2529/70; C10L 2270/023; C07C 2521/04

(57) ABSTRACT

A process for the catalytic alkylation of an olefin with an isoparaffin comprises contacting an olefin-containing feed with an isoparaffin-containing feed under alkylation conditions in a reaction zone containing a fixed bed of a solid acid catalyst comprising a crystalline microporous material of the MWW framework type, wherein the reaction zone contains at least 100 kg of the catalyst and the catalyst has a cycle length of at least 150 days.

### CROSS-REFERENCE TO RELATED APPLICATIONS

This application claims the benefit of U.S. Provisional Application No. 62/353,675, filed on Jun. 23, 2016, the entire contents of which is incorporated herein by reference.

### FIELD

The present disclosure relates to a process for isoparaffin-olefin alkylation.

### BACKGROUND

Alkylation is a reaction in which an alkyl group is added to an organic molecule. Thus an isoparaffin can be reacted with an olefin to provide an isoparaffin of higher molecular weight. Industrially, the concept depends on the reaction of a C₂ to C₅ olefin, normally 2-butene, with isobutane in the presence of an acidic catalyst to produce a so-called alkylate. This alkylate is a valuable blending component in the manufacture of gasoline due not only to its high octane rating but also to its sensitivity to octane-enhancing additives.

Industrial isoparaffin-olefin alkylation processes have historically used hydrofluoric or sulfuric acid catalysts under relatively low temperature conditions. The sulfuric acid alkylation reaction is particularly sensitive to temperature, with low temperatures being favored to minimize the side reaction of olefin polymerization. Acid strength in these liquid acid catalyzed alkylation processes is preferably maintained at 88 to 94 weight percent by the continuous addition of fresh acid and the continuous withdrawal of spent acid. The hydrofluoric acid process is less temperature sensitive and the acid is more easily recovered and purified.

A general discussion of sulfuric acid alkylation can be found in a series of three articles by L. F. Albright et al., “Alkylation of Isobutane with C₄ Olefins”, 27 Ind. Eng. Chem. Res., 381-397, (1988). For a survey of hydrofluoric acid catalyzed alkylation, see 1 Handbook of Petroleum Refining Processes 23-28 (R. A. Meyers, ed., 1986). An overview of the entire technology can be found in “Chemistry, Catalysts and Processes of Isoparaffin-Olefin Alkylation—Actual Situation and Future Trends, Corma et al., Catal. Rev.—Sci. Eng. 35(4), 483-570 (1993).

Both sulfuric acid and hydrofluoric acid alkylation share inherent drawbacks including environmental and safety concerns, acid consumption, and sludge disposal. Research efforts have, therefore, been directed to developing alkylation catalysts which are equally as effective as sulfuric or hydrofluoric acids but which avoid many of the problems associated with these two acids. In particular, research has been focused on the development of solid, instead of liquid, acid alkylation catalyst systems.

For example, U.S. Pat. No. 3,644,565 discloses alkylation of a paraffin with an olefin in the presence of a catalyst comprising a Group VIII noble metal present on a crystalline aluminosilicate zeolite having pores of substantially uniform diameter from about 4 to 18 angstrom units and a silica to alumina ratio of 2.5 to 10, such as zeolite Y. The catalyst is pretreated with hydrogen to promote selectivity.

However, the development of a satisfactory solid acid replacement for hydrofluoric and sulfuric acid has proved challenging. For example, U.S. Pat. No. 4,384,161 describes a process of alkylating isoparaffins with olefins to provide alkylate using a large-pore zeolite catalyst capable of absorbing 2,2,4-trimethylpentane, for example, ZSM-4, ZSM-20, ZSM-3, ZSM-18, zeolite Beta, faujasite, mordenite, zeolite Y and the rare earth metal-containing forms thereof, and a Lewis acid such as boron trifluoride, antimony pentafluoride or aluminum trichloride. The addition of a Lewis acid is reported to increase the activity and selectivity of the zeolite, thereby effecting alkylation with high olefin space velocity and low isoparaffin/olefin ratio. According to the '161 patent, problems arise in the use of solid catalysts alone in that they appear to age rapidly and cannot perform effectively at high olefin space velocity.

As a result commercial proposals for isoparaffin-olefin alkylation using solid acid catalysts have focused on reactor systems which allow for continuous or semi-continuous catalyst regeneration. Examples of such systems include fluidized and moving bed reactors, as well as swing bed systems where multiple reactors are oscillated between on-stream mode and regeneration mode. However, such reactor systems are expensive to construct and operate. Thus, there remains an unmet need for an improved isoparaffin-olefin alkylation process that is catalyzed by a solid acid catalyst but can be operated commercially in simple reactor systems.

### SUMMARY

According to the present disclosure, it has now been found that MWW framework type molecular sieves exhibit unexpectedly high activity, stability and selectivity for the production of alkylate when used as catalysts for isoparaffin-olefin alkylation with propylene-containing feeds. As a result, processes using these catalysts can be operated in fixed bed systems at commercially viable cycle lengths such that the need for multiple swing-bed reactors can be obviated.

Thus, in one aspect, the present disclosure provides a process for the catalytic alkylation of an olefin with an isoparaffin, the process comprising: contacting an olefin-containing feed with an isoparaffin-containing feed under alkylation conditions in a reaction zone containing a fixed bed of a solid acid catalyst comprising a crystalline microporous material of the MWW framework type, wherein the reaction zone contains at least 100 kg of the catalyst and the catalyst has a cycle length of at least 150 days.

### BRIEF DESCRIPTION OF THE DRAWINGS

FIG. 1 is a graph of butene conversion against the material balance (MB) number based on online GC analysis for (a) a sand blank, (b) an REX catalyst and (c) the MCM-49 catalyst of Example 1 in the alkylation of a premixed isobutane/butene feed at various temperatures according to the process of Example 2.

FIG. 2 is a graph of butene conversion against days on oil for the MCM-49 catalyst of Example 1 in the alkylation of a premixed isobutane/butene feed at various LHSV values according to the process of Example 2.

### DETAILED DESCRIPTION OF THE EMBODIMENTS

Disclosed herein is a process for isoparaffin-olefin alkylation, in which an olefin-containing feed is contacted with an isoparaffin-containing feed under alkylation conditions in a reaction zone containing a fixed bed of a solid acid catalyst comprising a crystalline microporous material of the MWW framework type. Surprisingly, it is found that the stability of the MWW framework type material is such that the process can be operated in a commercial scale fixed bed reactor, namely where the reaction zone contains at least 100 kg of the catalyst, with the catalyst exhibiting a cycle length of at least 150 days.

As used herein, the term “cycle length” of a specific catalyst refers to the number of days the catalyst can be continuously operated in a process for the alkylation of a 50:1 (volume/volume) mixture of isobutane and 2-butene at a temperature of 150° C. ° C., a pressure of 750 psig (5272 kPa-a) and an LHSV of 1.5-8 hr⁻¹ before the 2-butene conversion activity of catalyst decreases from an initial value, typically of at least 90 wt % butene conversion, to 50% of its initial value.

As used herein, the term “crystalline microporous material of the MWW framework type” includes one or more of:
molecular sieves made from a common first degree crystalline building block unit cell, which unit cell has the MWW framework topology. (A unit cell is a spatial arrangement of atoms which if tiled in three-dimensional space describes the crystal structure. Such crystal structures are discussed in the “Atlas of Zeolite Framework Types”, Fifth edition, 2001, the entire content of which is incorporated as reference);
molecular sieves made from a common second degree building block, being a 2-dimensional tiling of such MWW framework topology unit cells, forming a monolayer of one unit cell thickness, preferably one c-unit cell thickness;
molecular sieves made from common second degree building blocks, being layers of one or more than one unit cell thickness, wherein the layer of more than one unit cell thickness is made from stacking, packing, or binding at least two monolayers of MWW framework topology unit cells. The stacking of such second degree building blocks can be in a regular fashion, an irregular fashion, a random fashion, or any combination thereof; and
molecular sieves made by any regular or random 2-dimensional or 3-dimensional combination of unit cells having the MWW framework topology.

Crystalline microporous materials of the MWW framework type include those molecular sieves having an X-ray diffraction pattern including d-spacing maxima at 12.4±0.25, 6.9±0.15, 3.57±0.07 and 3.42±0.07 Angstrom. The X-ray diffraction data used to characterize the material are obtained by standard techniques using the K-alpha doublet of copper as incident radiation and a diffractometer equipped with a scintillation counter and associated computer as the collection system.

Examples of crystalline microporous materials of the MWW framework type include MCM-22 (described in U.S. Pat. No. 4,954,325), PSH-3 (described in U.S. Pat. No. 4,439,409), SSZ-25 (described in U.S. Pat. No. 4,826,667), ERB-1 (described in European Patent No. 0293032), ITQ-1 (described in U.S. Pat. No. 6,077,498), ITQ-2 (described in International Patent Publication No. WO97/17290), MCM-36 (described in U.S. Pat. No. 5,250,277), MCM-49 (described in U.S. Pat. No. 5,236,575), MCM-56 (described in U.S. Pat. No. 5,362,697), UZM-8 (described in U.S. Pat. No. 6,756,030), UZM-8HS (described in U.S. Pat. No. 7,713,513), UZM-37 (described in U.S. Pat. No. 7,982,084); EMM-10 (described in U.S. Pat. No. 7,842,277), EMM-12 (described in U.S. Pat. No. 8,704,025), EMM-13 (described in U.S. Pat. No. 8,704,023), MIT-1 (described by Luo et al in Chem. Sci., 2015, 6, 6320-6324), and mixtures thereof, with MCM-49 generally being preferred.

In some embodiments, the crystalline microporous material of the MWW framework type employed herein may be an aluminosilicate material having a silica to alumina molar ratio of at least 10, such as at least 10 to less than 50.

In some embodiments, the crystalline microporous material of the MWW framework type employed herein may be contaminated with other crystalline materials, such as ferrierite or quartz. These contaminants may be present in quantities ≦10% by weight, normally ≦5% by weight.

The above molecular sieves may be used in the alkylation catalyst without any binder or matrix, i.e., in so-called self-bound form. Alternatively, the molecular sieve may be composited with another material which is resistant to the temperatures and other conditions employed in the alkylation reaction. Such materials include active and inactive materials and synthetic or naturally occurring zeolites as well as inorganic materials such as clays and/or oxides such as alumina, silica, silica-alumina, zirconia, titania, magnesia or mixtures of these and other oxides. The latter may be either naturally occurring or in the form of gelatinous precipitates or gels including mixtures of silica and metal oxides. Clays may also be included with the oxide type binders to modify the mechanical properties of the catalyst or to assist in its manufacture. Use of a material in conjunction with the molecular sieve, i.e., combined therewith or present during its synthesis, which itself is catalytically active may change the conversion and/or selectivity of the catalyst. Inactive materials suitably serve as diluents to control the amount of conversion so that products may be obtained economically and orderly without employing other means for controlling the rate of reaction. These materials may be incorporated into naturally occurring clays, e.g., bentonite and kaolin, to improve the crush strength of the catalyst under commercial operating conditions and function as binders or matrices for the catalyst. The relative proportions of molecular sieve and inorganic oxide binder may vary widely. For example, the amount of binder employed may be as little as 1 wt %, such as at least 5 wt %, for example at least 10 wt %, whereas in other embodiments the catalyst may include up to 90 wt %, for example up 80 wt %, such as up to 70 wt %, for example up to 60 wt %, such as up to 50 wt % of a binder material.

In one embodiment, the solid acid catalyst employed in the present process is substantially free of any binder containing amorphous alumina. As used herein, the term “substantially free of any binder containing amorphous alumina” means that the solid acid catalyst used herein contains less than 5 wt %, such as less than 1 wt %, and preferably no measurable amount, of amorphous alumina as a binder. Surprisingly, it is found that when the solid acid catalyst is substantially free of any binder containing amorphous alumina, the activity of the catalyst for isoparaffin-olefin alkylation can be significantly increased, for example by at least 50%, such as at least 75%, even at least 100% as compared with the activity of an identical catalyst but with an amorphous alumina binder.

Feedstocks useful in the present alkylation process include at least one isoparaffin and at least one olefin. The isoparaffin reactant used in the present alkylation process may have from about 4 to about 8 carbon atoms. Representative examples of such isoparaffins include isobutane, isopentane, 3-methylhexane, 2-methylhexane, 2,3-dimethylbutane, 2,4-dimethylhexane and mixtures thereof, especially isobutane.

The olefin component of the feedstock may include at least one olefin having from 3 to 12 carbon atoms. Representative examples of such olefins include butene-2, isobutylene, butene-1, propylene, ethylene, hexene, octene, and heptene, merely to name a few. In some embodiments, the olefin component of the feedstock is selected from the group consisting of propylene, butenes, pentenes and mixtures thereof. For example, in one embodiment, the olefin component of the feedstock may include a mixture of propylene and at least one butene, especially 2-butene, where the weight ratio of propylene to butene is from 0.01:1 to 1.5:1, such as from 0.1:1 to 1:1. In another embodiment, the olefin component of the feedstock may include a mixture of propylene and at least one pentene, where the weight ratio of propylene to pentene is from 0.01:1 to 1.5:1, such as from 0.1:1 to 1:1.

Isoparaffin to olefin ratios in the reactor feed typically range from about 1.5:1 to about 100:1, such as 10:1 to 75:1, measured on a volume to volume basis, so as to produce a high quality alkylate product at industrially useful yields. Higher isoparaffin:olefin ratios may also be used, but limited availability of produced isoparaffin within many refineries coupled with the relatively high cost of purchased isoparaffin favor isoparaffin:olefin ratios within the ranges listed above.

Before being sent to the alkylation reactor, the isoparaffin and olefin may be treated to remove catalyst poisons e.g., using guard beds with specific absorbents for reducing the level of S, N, and/or oxygenates to values which do not affect catalyst stability activity and selectivity.

The present alkylation process is suitably conducted at temperatures from about 275° F. to about 700° F. (135° C. to 371° C.), such as from about 300° F. to about 600° F. (149° C. to 316° C.). Operating temperature typically exceed the critical temperature of the principal component in the feed. The term “principal component” as used herein is defined as the component of highest concentration in the feedstock. For example, isobutane is the principal component in a feedstock consisting of isobutane and 2-butene in isobutane:2-butene weight ratio of 50:1.

Operating pressure may similarly be controlled to maintain the principal component of the feed in the supercritical state, and is suitably from about 300 to about 1500 psig (2170 kPa-a to 10,445 kPa-a), such as from about 400 to about 1000 psig (2859 kPa-a to 6996 kPa-a). In some embodiments, the operating temperature and pressure remain above the critical value for the principal feed component during the entire process run, including the first contact between fresh catalyst and fresh feed.

Hydrocarbon flow through the alkylation reaction zone containing the catalyst is typically controlled to provide a total liquid hourly space velocity (LHSV) sufficient to convert about 99 percent by weight of the fresh olefin to alkylate product. In some embodiments, olefin LHSV values fall within the range of about 0.01 to about 10 hr⁻¹.

The present isoparaffin-olefin alkylation process is conducted in fixed bed reactor, which may include one or a plurality of reaction zones, connected in series or in parallel, such that, when operated on a commercial scale, the total amount of catalyst in the reaction zone(s) is at least 100 kg, such as at least 500 kg, for example at least 1,000 kg. Even in a reactor system without swing bed capability (that is having multiple reactors which can be oscillated between on-stream mode and regeneration mode) it is found that the present catalyst exhibits cycle lengths (as defined above) in excess of 150 days, such as in excess of 200 days, even in excess of 250 days. Regeneration can then be effected by removing heavy hydrocarbon either by purging the catalyst with inert gas at high temperature or using oxygen for burning the heavies and free the accessibility to the acid sites. A suitable regeneration procedure involves heating the spent catalyst at 200 to 550° C. under gas (air or inert gas) flow rate >100 cc/min. for 20 minutes to a few hours. Gas analysis from the reactor is performed to monitor the regeneration process. Regeneration can be done in situ or ex situ.

The product composition of the isoparaffin-olefin alkylation reaction described herein is highly dependent on the reaction conditions and the composition of the olefin and isoparaffin feedstocks. In any event, the product is a complex mixture of hydrocarbons, since alkylation of the feed isoparaffin by the feed olefin is accompanied by a variety of competing reactions including cracking, olefin oligomerization and further alkylation of the alkylate product by the feed olefin. For example, in the case of alkylation of isobutane with C₃-C₅ olefins, particularly 2-butene, the product may comprise about 20 wt % of C₅-C₇ hydrocarbons, 60-65 wt % of octanes and 15-20 wt % of C₉+ hydrocarbons. Moreover, using an MWW type molecular sieve as the catalyst, it is found that the process is selective to desirable high octane components so that, in the case of alkylation of isobutane with C₃-C₅ olefins, the C₆ fraction typically comprises at least 40 wt %, such as at least 70 wt %, of 2,3-dimethylbutane and the C₈ fraction typically comprises at least 50 wt %, such as at least 70 wt %, of 2,3,4 and 2,33 and 2,2,4-trimethylpentane.

The product of the isoparaffin-olefin alkylation reaction is conveniently fed to a separation system, such as a distillation train, to recover the C5-9-fraction for use as a gasoline octane enhancer. Depending on alkylate demand, part of all of the remaining C₁₀₊ fraction can be recovered for use as a distillate blending stock or can be recycled to the alkylation reactor to generate more alkylate. In particular, it is found that MWW type molecular sieves are effective to crack the C₉₊ fraction to produce light olefins and paraffins which can react to generate additional alkylate product and thereby increase overall alkylate yield.

The invention will now be more particularly described with reference to the following non-limiting Examples and the accompanying drawings.

### Example 1 Preparation of 80 wt % MCM-49/20 wt % Alumina Catalyst

80 parts MCM-49 zeolite crystals are combined with 20 parts pseudoboehmite alumina, on a calcined dry weight basis. The MCM-49 and pseudoboehmite alumina dry powder are placed in a muller or a mixer and mixed for about 10 to 30 minutes. Sufficient water and 0.05% polyvinyl alcohol are added to the MCM-49 and alumina during the mixing process to produce an extrudable paste. The extrudable paste is formed into a 1/20 inch quadralobe extrudate using an extruder. After extrusion, the 1/20th inch quadralobe extrudate is dried at a temperature ranging from 250° F. to 325° F. (121 to 163° C.). After drying, the dried extrudate is heated to 1000° F. (538° C.) under flowing nitrogen. The extrudate is then cooled to ambient temperature and humidified with saturated air or steam.

After humidification, the extrudate is ion exchanged with 0.5 to 1 N ammonium nitrate solution. The ammonium nitrate solution ion exchange is repeated. The ammonium nitrate exchanged extrudate is then washed with deionized water to remove residual nitrate prior to calcination in air. After washing the wet extrudate, it is dried. The exchanged and dried extrudate is then calcined in a nitrogen/air mixture to a temperature 1000° F. (538° C.).

### Example 2 Testing in Isobutane/2-Butene Alkylation

The catalyst of Example 1 was compared with a sand blank and a commercial REX catalysts in the alkylation testing of a mixture of isobutane and 2-butene having the following composition by weight:

| | |
|---|---|
| 1-butene | 0.01% |
| Cis-2-butene | 1.25% |
| Trans-2-butene | 1.19% |
| Iso-C₄= | 0.00% |
| Iso-butane | 97.37% |
| n-butane | 0.23% |

The reactor used in these experiments comprised a stainless steel tube having an internal diameter of ⅜ in, a length of 20.5 in and a wall thickness of 0.035 in. A piece of stainless steel tubing 8¾ in. long×⅜ in. external diameter and a piece of ¼ inch tubing of similar length were positioned in the bottom of the reactor (one inside of the other) as a spacer to position and support the catalyst in the isothermal zone of the furnace. A ¼ inch plug of glass wool was placed at the top of the spacer to keep the catalyst in place. A ⅛ inch stainless steel thermo-well was placed in the catalyst bed, long enough to monitor temperature throughout the catalyst bed using a movable thermocouple. The catalyst is loaded with a spacer at the bottom to keep the catalyst bed in the center of the furnace's isothermal zone.

The catalyst was then loaded into the reactor from the top. The catalyst bed typically contained about 4 gm of catalyst sized to 14-25 mesh (700 to 1400 micron) and was 10 cm. in length. A ¼ in. plug of glass wool was placed at the top of the catalyst bed to separate quartz chips from the catalyst. The remaining void space at the top of the reactor was filled with quartz chips. The reactor was installed in the furnace with the catalyst bed in the middle of the furnace at the pre-marked isothermal zone. The reactor was then pressure and leak tested typically at 300 psig (2170 kPa-a).

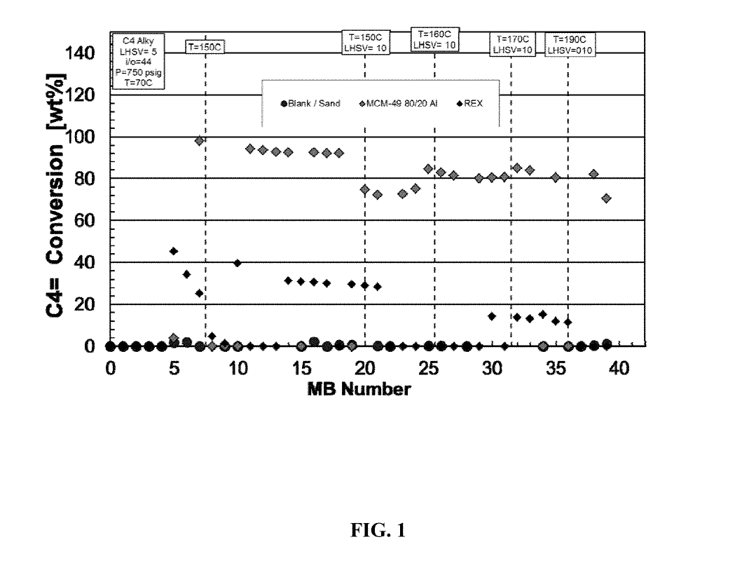

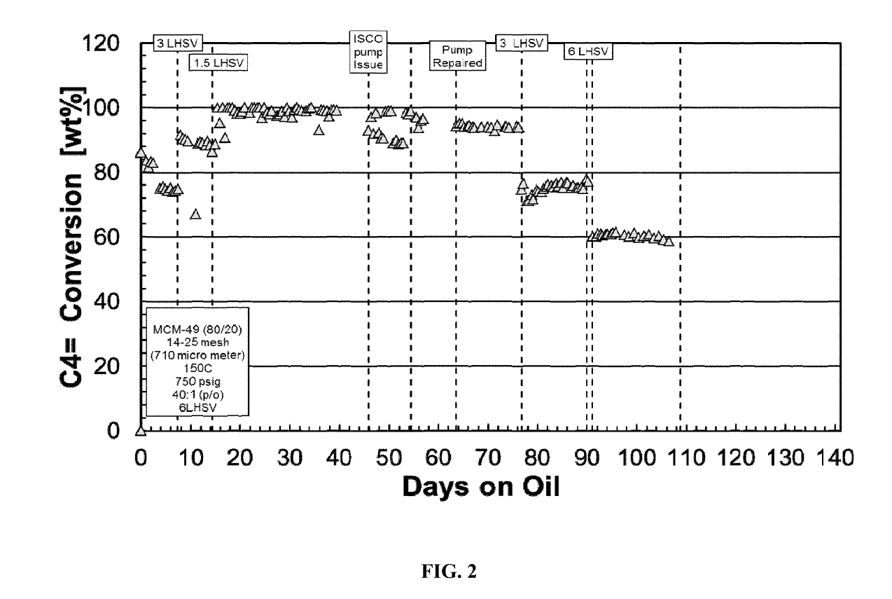

500 cc ISCO syringe pumps were used to introduce the feed to the reactor. Two ISCO pumps were used for pumping the iso-butane (high flow rate 15-250 cc/hr) and one ISCO pump for pumping 2-butene (0.3-5 cc/hr). A Grove “Mity Mite” back pressure controller was used to control the reactor pressure typically at 750 psig (5272 kPa-a). On-line GC analyses were taken to verify feed and the product composition. The products exiting the reactor flowed through heated lines routed to GC then to three cold (5-7° C.) collection pots in series. The non-condensable gas products were routed through a gas pump for analyzing the gas effluent. Material balances were taken at 24 hr intervals. Samples were taken for analysis. The material balance and the gas samples were taken at the same time while an on-line GC analysis was conducted for doing material balance. The results of the catalytic testing are shown in FIGS. 1 and 2.

FIG. 1 demonstrates that the MCM-49 catalyst of Example 1 exhibited significantly higher butene conversion activity and stability than the REX catalyst at all the conditions tested, namely an LHSV of 4 to 10 hr⁻¹ and a reaction temperature between 150° C. and 190° C. The sand blank exhibited essentially no butene conversion activity.

FIG. 2 shows the effect on the butene conversion activity of the MCM-49 catalyst of Example 1 of varying the LHSV between 1.5 to 6 hr⁻¹ at a constant reaction temperature of 150° C. over a prolonged period of 100 days on stream. It will be seen that, although the butene conversion showed a stepwise change when the LHSV was varied, the conversion then remained substantially constant until the next LHSV change. In one case, after about 40 days on stream, a gradual reduction in butene conversion was seen but this was traced to a pump problem and the activity was restored when the pump problem was fixed at around 60 days on stream.

While the present invention has been described and illustrated by reference to particular embodiments, those of ordinary skill in the art will appreciate that the invention lends itself to variations not necessarily illustrated herein. For this reason, then, reference should be made solely to the appended claims for purposes of determining the true scope of the present invention.

1. A process for the catalytic alkylation of an olefin with an isoparaffin, the process comprising: contacting an olefin-containing feed with an isoparaffin-containing feed under alkylation conditions in a reaction zone containing a fixed bed of a solid acid catalyst comprising a crystalline microporous material of the MWW framework type, wherein the reaction zone contains at least 100 kg of the catalyst and the catalyst has a cycle length of at least 150 days.

2. The process of claim 1, wherein the solid acid catalyst is substantially binder-free.

3. The process of claim 1, wherein the solid acid catalyst comprises an inorganic oxide binder.

4. The process of claim 3, wherein the inorganic oxide binder comprises alumina.

5. The process of claim 3, wherein the inorganic oxide binder is substantially free of amorphous alumina.

6. The process of claim 3, wherein the inorganic oxide binder comprises silica.

7. The process of claim 1, wherein the crystalline microporous material of the MWW framework type is selected from the group consisting of MCM-22, PSH-3, SSZ-25, ERB-1, ITQ-1, ITQ-2, MCM-36, MCM-49, MCM-56, EMM-10, EMM-12, EMM-13, UZM-8, UZM-8HS, UZM-37, MIT-1, and mixtures thereof.

8. The process of claim 1, wherein the crystalline microporous material comprises MCM-49.

9. The process of claim 1, wherein the MWW framework type material contains up to 10% by weight of impurities of other framework structures.

10. The process of claim 1, wherein the olefin-containing feed comprises at least one C₃ to C₁₂ olefin.

11. The process of claim 1, wherein the olefin-containing feed is selected from the group consisting of propylene, butenes, pentenes and mixtures thereof.

12. The process of claim 1, wherein the isoparaffin-containing feed comprises at least one C4 to C₈ isoparaffin.

13. The process of claim 1, wherein the isoparaffin-containing feed comprises isobutane.

14. The process of claim 1, wherein at least one of the olefin-containing feed and the isoparaffin-containing feed is pretreated to remove impurities prior to the contacting step.

15. The process of claim 1, wherein the alkylation conditions include a temperature at least equal to the critical temperature of the principal component of the combined olefin-containing feed and isoparaffin-containing feed and pressure at least equal to the critical pressure of the principal component of the combined olefin-containing feed and isoparaffin-containing feed.
